(19) United States
(12) Patent Application Publication
(10) Pub. No.: US 2011/0054462 A1
(43) Pub. Date: Mar. 3, 2011

(54) Electrosurgical instrument with multi-function handpiece

(76) Inventor: Alan G. Ellman, Oceanside, NY (US)

(21) Appl. No.: 12/583,870

(22) Filed: Aug. 28, 2009

(51) Int. Cl. A61B 18/14; A61B 18/18

(52) U.S. Cl. 606/33; 606/42

(57) ABSTRACT

An electrosurgical instrument with which a single handpiece can be connected that can be used for cutting and for coagulation without touching the instrument after the front panel settings have been made. The handpiece, of the extendable electrode type, comprises essentially two cables one terminating in a male connector configured to mate to the bipolar output connector of the electrosurgical unit, and a second terminating in a male connector configured to mate with the monopolar output connector of the electrosurgical unit. The output connectors can be selectively activated.

### RELATED APPLICATION

Commonly-owned U.S. application Ser. No. 11/090,858, filed Mar. 28, 2005 (hereinafter the '858 application).

Commonly-owned U.S. application Ser. No. 12/228,838, filed Aug. 18, 2008 (hereinafter the '838 application).

The invention is directed to an electrosurgical instrument, and in particular to a novel electrosurgical instrument comprising a handpiece connected to an electrosurgical unit such that the same handpiece with the same electrode can perform several electrosurgical functions normally requiring several handpieces or at least several different electrodes.

### BACKGROUND OF INVENTION

The '858 application describes an electrosurgical instrument well known and widely used in the medical field. It offers the capability of precision cutting and coagulation with electrosurgical currents preferably in the megacycle range using an RF probe or handpiece with, for example, needle, ball, or loop electrodes in a monopolar operating mode or with a forceps or other electrode in a bipolar operating mode. Such an instrument now sold by Elliquence, LLP of Oceanside, New York, as illustrated in the '858 application (also published as EP1707147A1) provides on its front panel a female connector (also called receptacle) for receiving the male plug of a cable-connected monopolar handpiece, a connector for receiving a ground or neutral plate, as well as a female connector for receiving the plug of a cable-connected bipolar electrode. Other forms of such an instrument are described in U.S. Pat. Nos. 5,954,686, 6,652,514, and 7,094,231, all of whose contents are incorporated herein by reference. The instruments described in those patents and typical of others in the art are characterized by different modes and sub-modes of operation. For example, the instruments have five possible operating modes, a cutting mode, separable into CUT and CUT/COAG (sometimes called blend) sub-modes, and a coagulation mode, separable into HEMO, FULGURATE, and BIPOLAR sub-modes. In the instruments described in these patents and applications, different frequencies serve as the carriers for the cutting and coagulation actions (thought this is not essential to the present invention), and the carriers are modulated by different waveforms to form the variety of electrosurgical currents used in the different surgical procedures. The instrument described in the '858 application provides the further improvement of improved control software to boost the operational applications to include a more intense coagulating mode known as BIPOLAR TURBO. To operate the instrument a triple footswitch control is provided for controlling monopolar and two bipolar applications, and/or a three-button fingerswitch handpiece is also provided for separately controlling three monopolar modes, respectively—these two control accessories can thus control and activate any desired selected modes with the instrument's preset output power capabilities. As described in detail in the referenced patents and applications, operating a footswitch or a fingerswitch (by the user-surgeon), turns on an RF generator to supply the carrier frequencies and then modulates the carrier with the selected waveform, and supplies the thus produced RF electrosurgical currents to either the selected monopolar output connector or the bipolar output connector.

In a typical surgical setting using such electrosurgical instruments, a surgeon may connect a monopolar handpiece with a cutting electrode to the monopolar connector on the instrument front panel, and then connect a second handpiece with a HEMO electrode to the bipolar connector. During the surgical procedure, the surgeon could first use the monopolar handpiece connected to the monopolar output connector while the monopolar instrument setting is in its cutting mode to perform a desired cutting procedure and then change to the second handpiece connected to the bipolar output connector to coagulate any bleeding blood vessels with the instrument setting in its coagulation mode. With the instrument settings set as indicated, the appropriate footswitch can be used to activate the instrument being used by directing as explained above the selected modulated carrier to the selected output connector. The reader can acquire any additional information desired by referencing the referenced patents and applications.

The important point is that at least two sets of cables connected between the instrument and the several handpieces are required for the surgeon carry out the steps of the procedure. In addition, often a source of suction is desirable at the surgical site to remove vapors and undesired fluids interfering with the surgeon's view of the surgical site. So, an additional cable or duct will be present requiring handling by an associate during the procedure.

### SUMMARY OF INVENTION

A principal object of the invention is an electrosurgical instrument of the type described with which a single handpiece can be connected that can be used for cutting and for coagulation without touching the instrument after the front panel settings have been made.

A major advantage results in that the surgeon does not have to switch handpieces during a typical procedure and only a single set of cables is present between the instrument and the handpiece(s).

This object is achieved in accordance with one aspect of the invention by taking advantage of a novel handpiece design originally designed for coagulation procedures and which relies on the active end of the electrode being capable of extension and retraction from within a tubular body under control of a surgeon manipulating a squeezable handle attached to the handpiece.

U.S. Pat. No. 6,231,571 (hereinafter the '571 patent) describes such a handpiece provided with a spring-biased handle that when squeezed and relaxed extends and retracts selectively a bipolar electrode from within a tubular member. The active end of the electrode is in the form of spaced half-balls between which electrosurgical currents can be generated when bipolar RF power is applied to the handpiece. The handpiece has also been applied to extend tips of a bipolar electrode typically used for hemostasis similar to forceps tips whose ends can be closed over a bleeder and the bleeder sealed off by applying hemo electrosurgical currents to the electrode.

A feature of the invention is to connect essentially two cables to an extendable handpiece of the type described, a first cable terminating in a male connector configured to mate to the bipolar output connector of the electrosurgical unit, and a second cable terminating in a male connector configured to mate with the monopolar output connector of the electrosurgical unit. The first cable is wired to connect the active terminals of the bipolar output connector to each of the active electrode tips. The second cable is wired to connect the active terminal of the monopolar output connector to one of the active electrode tips. As a result, when the bipolar generator is activated to supply bipolar currents to the bipolar output connector, bipolar electrosurgical currents are generated between the active electrode tips, and the surgeon can then coagulate blood vessels or ablate or modulate tissue as needed. Yet, when the monopolar generator is activated to supply monopolar currents to the monopolar output connector, monopolar electrosurgical currents are generated at least at one of the active electrode tips, and the surgeon can then perform cutting operations with the same handpiece as needed using the edge of the active electrode tip. By allowing the electrode tips to come together, by the surgeon relaxing his or her grip on the handle, then the second electrode tip also becomes active due to electrical contact with the first tip and the joint edges can be used to perform cutting or cut/coag operations with monopolar currents.

Another feature of the invention is to provide a channel in the handpiece tubular member and connect a suction fitting to the handpiece so that suction can be provided at the surgical site. This then omits the need for separate suction apparatus and the additional hoses. Thus, the handpiece combines monopolar and bipolar action together with suction to remove noxious odors.

Still another feature is to combine the fingerswitch feature together with the sliding action of the '571 patent into a pencil shaped handpiece without the handles. In this aspect of the invention, the fingerswitches would be connected to select monopolar or bipolar activation, as had been implemented in the referenced applications, and the slide would be connected to the electrodes so as to extend and retract the electrodes as described in the '838 application to select cutting or coagulation.

All of the features of the electrosurgical unit described in the '858 application can be employed with the present invention, including two control accessories (footswitch and fingerswitches) to control and activate any desired selected modes with the instrument's preset output power capabilities, all output parameters can be adjusted prior to the surgical procedure, and the electrosurgical instrument can be designed to operate with manually-chosen operating conditions, and also incorporates one or more sets of stored or preset operating modes and conditions that allows the surgeon to select a particular set customized for the particular procedure to be carried out.

The various features of novelty which characterize the invention are pointed out with particularity in the claims annexed to and forming a part of this disclosure. For a better understanding of the invention, its operating advantages and specific objects attained by its use, reference should be had to the accompanying drawings and descriptive matter in which there are illustrated and described the preferred embodiments of the invention, like reference numerals or letters signifying the same or similar components.

### SUMMARY OF THE DRAWINGS

In the drawings:

FIG. 1A is a schematic diagram of the front of a known electrosurgical unit;

FIG. 1B is a block diagram of the electronics of the known electrosurgical unit of FIG. 1A;

FIG. 2 is a schematic diagram of the electrosurgical unit of FIG. 1 shown connected to a footswitch and handpiece in accordance with the invention;

FIG. 3 illustrates the separation of the electrode tips when the handpiece handle is squeezed of the handpiece of FIG. 2;

FIG. 4 is a schematic circuit diagram illustrating one way of interconnecting the connector cables of the instrument of FIG. 2;

FIG. 5 is a schematic diagram of a handpiece with fingerswitches that can be used in the system in accordance with the invention;

FIG. 6 shows another form of handpiece useful in the invention;

FIG. 7 is a schematic diagram of another form of handpiece that can be used in the system in accordance with the invention;

FIG. 8 is an enlarged view of the electrode tips of FIG. 7 when separated.

FIG. 9 is a schematic diagram of still another form of handpiece that can be used in the system in accordance with the invention;

FIG. 10 is an enlarged view of the electrode tips of FIG. 9 when abutting;

FIG. 11 is an enlarged view of the electrode tips of FIG. 9 when separated.

### DETAILED DESCRIPTION OF PREFERRED EMBODIMENTS

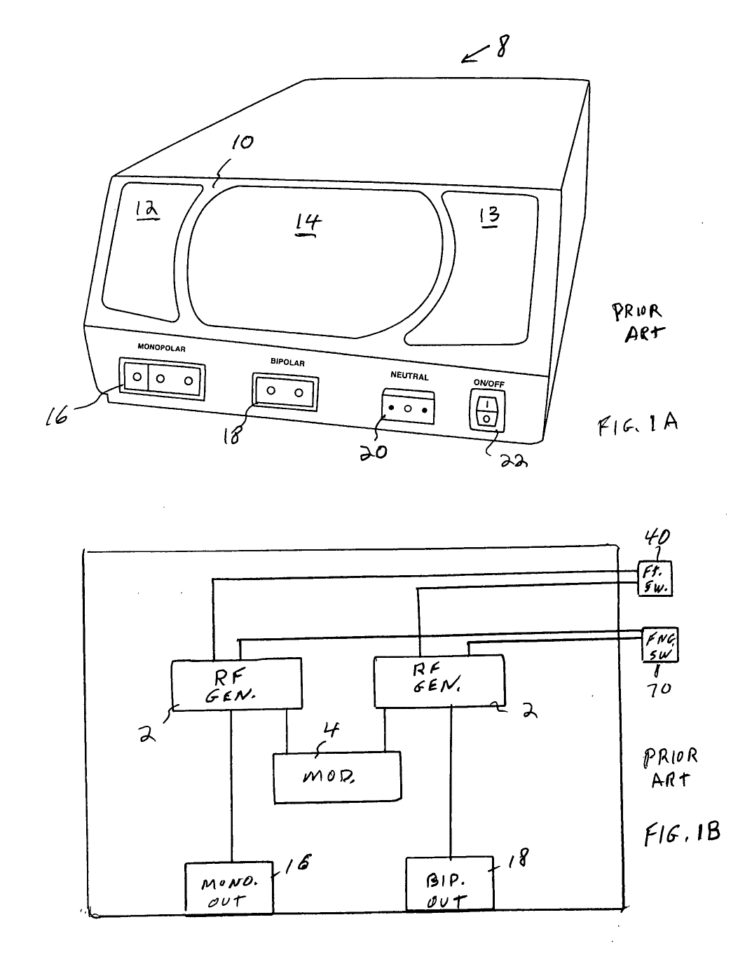

FIGS. 1A and 1B illustrate schematically a known electrosurgical unit available commercially from Elliquence, LLP of Oceanside, New York. It comprises a system unit 8 having a box-like housing comprising at the front a control panel 10 for the instrument. The control panel includes touch switches (not shown) in the area 12 for toggling among the five cutting or coagulation or hemo modes and touch switches (not shown) in the area 13 for controlling the power output by increasing or decreasing in steps the power, the values selected being displayed in the center area 14. At the bottom are output female connectors 16, 18, 20 for plugging in, respectively, at the left, a monopolar handpiece with or without fingerswitches; at the center, a bipolar handpiece; and at the right a single or split neutral plate. An on-off power switch 22 is at the far right. A connector (not shown) is provided at the side for receiving a triple or 3-way footswitch. The electronics include RF generators 2, a modulator 4 for generating the waveforms for the different currents, and wiring connecting the modulated RF outputs to the output connectors 16, 18.

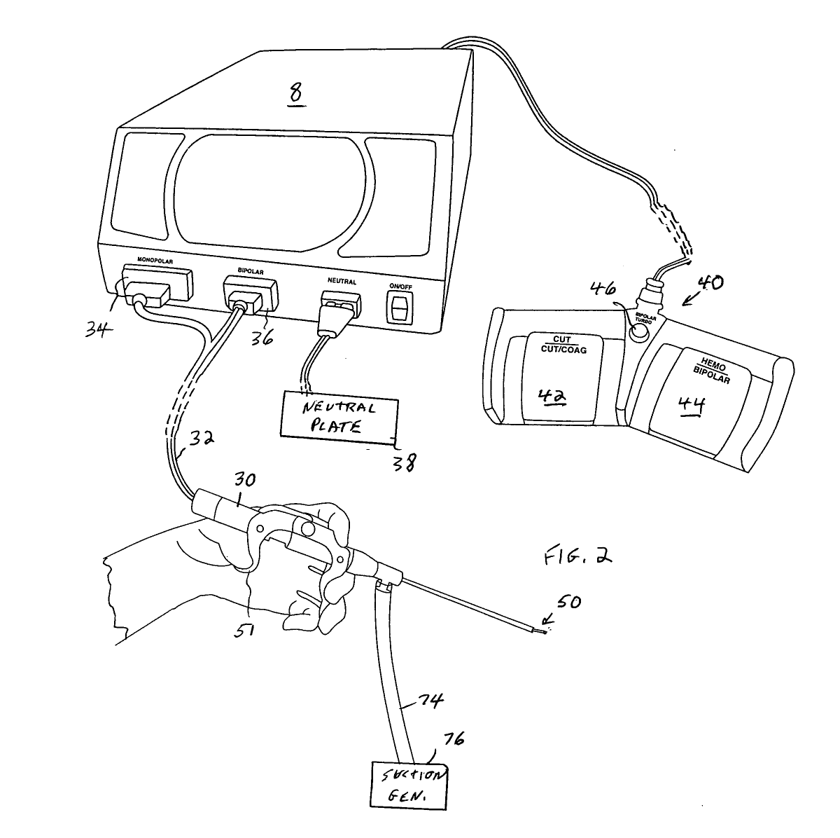

In the system in accordance with the invention, in place of separate monopolar and bipolar handpieces, each separately plugged into its respective output connector 16, 18, a single handpiece 30 is provided with one or two cables terminated in male connectors for mating with both of the output connectors. This is illustrated in FIG. 2 which shows the handpiece 30 connected by a cable 32 dividing into a male plug 34 for plugging into the monopolar output connector 16, and a male plug 36 for plugging into the bipolar output connector bipolar output connector 18. It also shows a neutral plate 38 plugged into the neutral connector 20. A conventional triple footswitch 40 is also shown connected to the unit 8. The left footswitch section 42 when depressed supplies RF power to the monopolar connector 16. The right section 44 of the footswitch when depressed supplies RF power to the bipolar connector 18. This unit also provides bipolar turbo-hemo action, to the bipolar output connector, when a center footswitch button 46 is depressed. These actions in response to footswitch activation is conventional and is implemented by hardware or software as is well known in the art. Software control ensures only one of the unit's output connectors is activated at a time, essentially an Exclusive-Or arrangement. A particular appropriate unit is that sold by Elliquence, LLC of Oceanside, New York under the name “Surgi-Max”.

Which output connector is activated is determined by which of the triple footswitches is activated. Similar action occurs when fingerswitches are provided on the handpiece, one of which when activated causes the unit to supply desired electrosurgical currents to one or the other of the output connectors. As shown in FIG. 1A, the monopolar connector 16 typically has 3 terminals, two of which are used to supply fingerswitch control signals to the unit, and the third of which is the active terminal, meaning it is the one supplied with an RF voltage at a certain polarity when the correct footswitch or fingerswitch is activated. In the monopolar mode, the return is provided by the neutral plate 38 coupled to the patient typically at ground potential, the electrosurgical currents flowing between the electrode at, say, a positive voltage and the neutral plate at the negative polarity. The bipolar connector 18 has 2 terminals, one of which is supplied with an RF voltage at a positive polarity and the other with the opposite negative polarity voltage, the electrosurgical currents flowing between the two bipolar electrode tips when the voltage is applied to the handpiece. The Elliquence Surgi-Max unit provides to the monopolar connector RF electrosurgical currents at a frequency of about 4 MHz, and to the bipolar connector RF electrosurgical currents at a frequency of about 2 MHz. These different frequencies are not as such significant to the present invention. As with other electrosurgical units, the RF currents supplied to the monopolar connector 16, typically used for cutting or for cutting/coagulating, are modulated differently than those supplied to the bipolar connector 18 typically used for hemostasis. From the present invention standpoint, the important consideration is that a terminal of each of the output connectors when energized is active, in the sense that it is supplied with an RF voltage, which may be and preferably is of the same polarity.

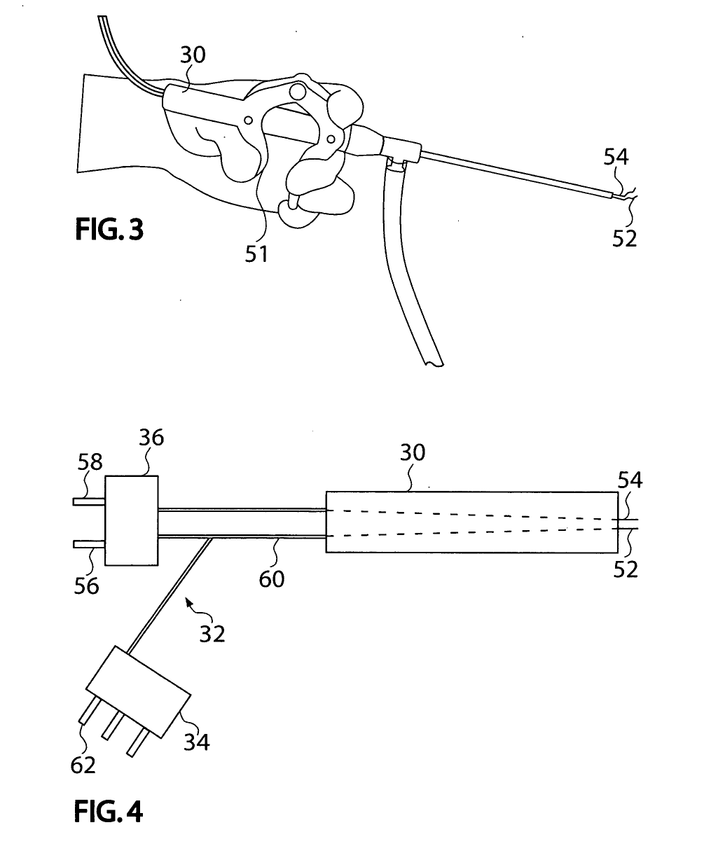

The handpiece 30 illustrated in FIG. 2 is preferably of the type described in the '571 patent, which is of the bipolar type with electrode tips that can be extended and retracted. One simple form can be of two parallel tips 50 as shown in FIG. 2. When the handle 51 is squeezed, as shown in FIG. 3, the electrode tips 52, 54 extend further and separate. FIG. 4 schematically illustrates the wiring of the handpiece 30 and cable 32. Each electrode 52, 54, as is common for bipolar handpieces, is wired internally through the handpiece housing and via the cable 32 to each of the bipolar connector pins 56, 58 respectively. A novel feature of the invention is the addition of a second male connector 34 to that same cable. This second male connector should be configured to mate with the standard female monopolar connector 16 on the unit's front panel. Now, when the female monopolar connector of the unit is of the type disclosed above, with one active terminal, then in accordance with the invention, the cable wire 32 connecting the pin 62 that will engage the socket 16 of the active terminal is connected to the cable wire of one of the active bipolar terminals, in this case the pin 56 connected to the electrode 52, which is preferably but not necessarily of the same polarity. Thus, if the monopolar active terminal when energized is at a positive polarity, then the interconnection is made to the same polarity bipolar terminal.

In operation, when the bipolar output connector 18 is energized, then RF bipolar currents are supplied from the bipolar output connector 18 by way of the plug 36 and handpiece cable 60 to the separate electrode tips 52, 54 in the usual way, and the tips, manipulated by the user squeezing the handle 51, can be used to seal off bleeders. When the monopolar output connector 16 is energized, then RF electrosurgical currents are supplied via the plug 34 to one of the internal wires 60 of the handpiece connected to the electrode 52, and that energized electrode 52 can be used to perform a cutting action with monopolar currents. Preferably, when the handle 51 is manipulated to allow the electrode tips 52, 54 to touch, without tissue between them, then the active tip 52 energizes the other tip 54 and both tips are now at the same active potential and together can be used to supply electrosurgical cutting action.

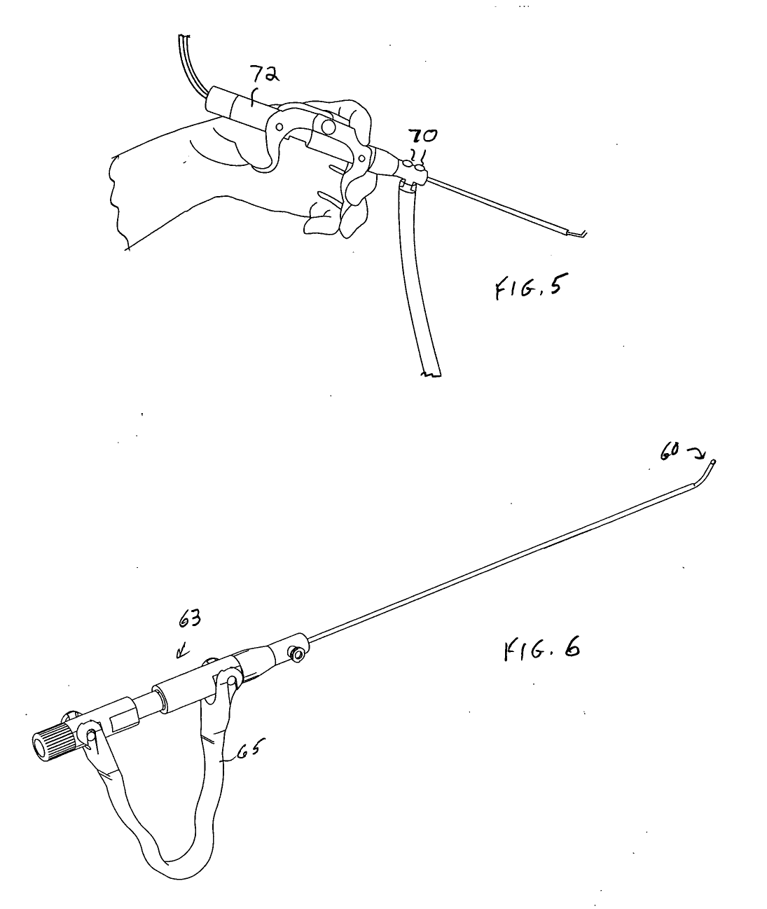

Different shapes of tips are possible with different kinds of similarly functioning handpieces. For example, in FIG. 3, the tips 52, 54 also extend upward when extended. FIG. 6 shows a handpiece 63 with a different kind of handle 65 but operating similarly, and with electrodes 60 that are fixed together. In this case, bipolar action occurs across the face of the active electrode tips when the bipolar currents are supplied. When monopolar currents are supplied, then one of the tips becomes active and can function to perform a cutting or combined cutting-coagulation action.

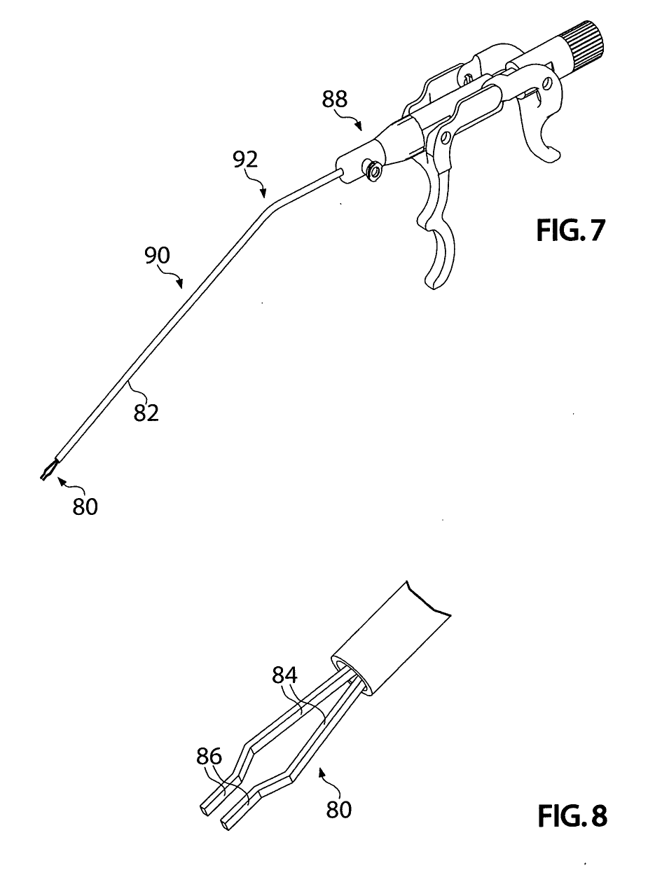

FIGS. 7 and 8 show another tip geometry that can be used, described in the '838 application. This electrode 80 is characterized by the configuration of the tips of the bipolar electrode ends, which are formed into flat opposing surfaces, and the relationship of their connecting links to the tubular member 82 which surrounds then. The connecting links 84 are configured such that, as the handles are released, when they first are forced to close as they withdraw into the tubular member, the distal ends of the electrode tips touch first. As they continue to withdraw into the tubular member and to close, the flat surfaces 86 are pressed up against one another. This action ensures that any tissue grasped as the tips close is held securely in the tips and not squeezed out. But it was also recognized that when the tips 80 are fully extended without any tissue or other material between the tips, the tips are pressed closely against one another and actually form a single flat electrode shape that functions extremely well as an electrosurgical cutting electrode.

Thus, with the tips 80 fully extended with its ends pressed up against one another, the application of monopolar electrosurgical currents will allow the surgeon to perform precise cutting operations with the adjacent tips, and when the handle is slowly released, the tips 80 separate and then can be used to embrace a bleeder and seal off that bleeder when bipolar electrosurgical currents are applied to the same handpiece 88. This tip arrangement, shown enlarged in FIG. 8, in which the shape of the tips 86—narrow rectangles with broader facing surfaces—allows the narrow edges of the tips when abutting to serve the cutting function, whereas when the tips are separated the broader facing surfaces are well suited for grasping bleeders to seal them off in the bipolar mode. The invention is not limited to the tip shapes shown. Other tip shapes will also prove useful even in the form of adjacent loops configured to preferably provide a narrow surface when abutting for cutting and a reasonably broader surface when separated for hemo. FIG. 7 also shows that the elongated tube 90 housing the electrodes need not be straight but can also include a bend 92 about one-third along its length which sometimes helps visibility of the surgical site in certain positions of the handpiece.

While it is preferred that a handpiece of the type described in FIGS. 7 and 8 is used, as the tip shape is particularly well suited for monopolar use when the tips are together and for bipolar use when the tips are separated, other handpieces can be substituted. For example, a conventional forceps electrode can be used. The typical forceps electrode has a bipolar connector and the surgeon manipulates the forceps sides to seal bleeders, but with such a handpiece provided with the interconnected second monopolar plug as described above, the surgeon can obtain monopolar cutting when the forceps tips are closed.

FIG. 2 illustrates a footswitch 40 with 3 positions for providing monopolar activation, bipolar activation, and turbo hemo activation as described in the '858 application. The invention is not limited to such activation. For example, fingerswitches 70 on the handpiece 72 as illustrated in FIG. 5 can be used to selectively activate the monopolar or bipolar output connector. This function is readily obtained by software, which responds to the signals supplied by the fingerswitches to the unused two terminals of the monopolar connector and these signal using in turn well known software controls.

The invention is not limited to a 3-position footswitch. For example, a 5-position footswitch would be quite useful, including separate monopolar cut, blend, and hemo positions, as well as separate bipolar hemo and turbo positions. All are easily implemented by software responding to signals from the footswitches or if desired fingerswitches.

It is also noted that a suction hose 74 is connected to the handpiece in FIG. 2. The handpiece interior is hollow or provided with a separate tube, and thus the suction from a suction source 76 will be accessible at the distal end of the handpiece adjacent the tips and can easily remove undesired vapors and other materials as needed during the procedure.

Many applications can make use of the system of the invention. Particularly useful medical procedures would involve brain surgery and what is now called keyhole surgery, with or without endoscopic assistance and viewing, and spinal surgery, though the system of the invention is also useful wherever electrosurgery procedures can be used.

The RF power generating circuitry may be of the well known tube-type described in U.S. Pat. No. 3,730,188, whose contents are herein incorporated by reference, which is capable of generating a fully-rectified, filtered RF current for cutting, a full-wave rectified current for combining cutting and coagulation, and a half-wave rectified current for coagulation. Alternatively, the RF power generating circuitry can be of the well-known solid-state type capable of generating the same kinds of waveforms. Those skilled in the art will know how to produce the turbo mode modulation from the description given. The RF circuitry, as such, is not part of the present invention, as such circuits are well-known in the prior art. In this particular example, the RF circuitry provides two different frequencies of operation, a first high frequency in the range of 3.8-4.0 MHz, 4.0 being preferred, and a second high frequency in the range of 1.7-2.0 MHz, which is easily obtained by providing a known RF generator that provides outputs at double these first and second higher frequencies and providing a simple known divide-by-two circuit for obtaining dual outputs at the first or second frequencies, respectively. Both outputs can be separately amplified and processed and made available at the console's output connectors depending on the switches activated. Aspects of the present invention are not limited to the dual-frequency output operation.

After the modulated carrier has been generated, it is processed through a standard driver, a transformer, and a power amplifier controlled by a bias signal and whose input can be monitored for safety's sake by a power tester circuit under control of a μ-controller. The output connectors are electrically isolated as by isolation transformers as described in the referenced applications/patents.

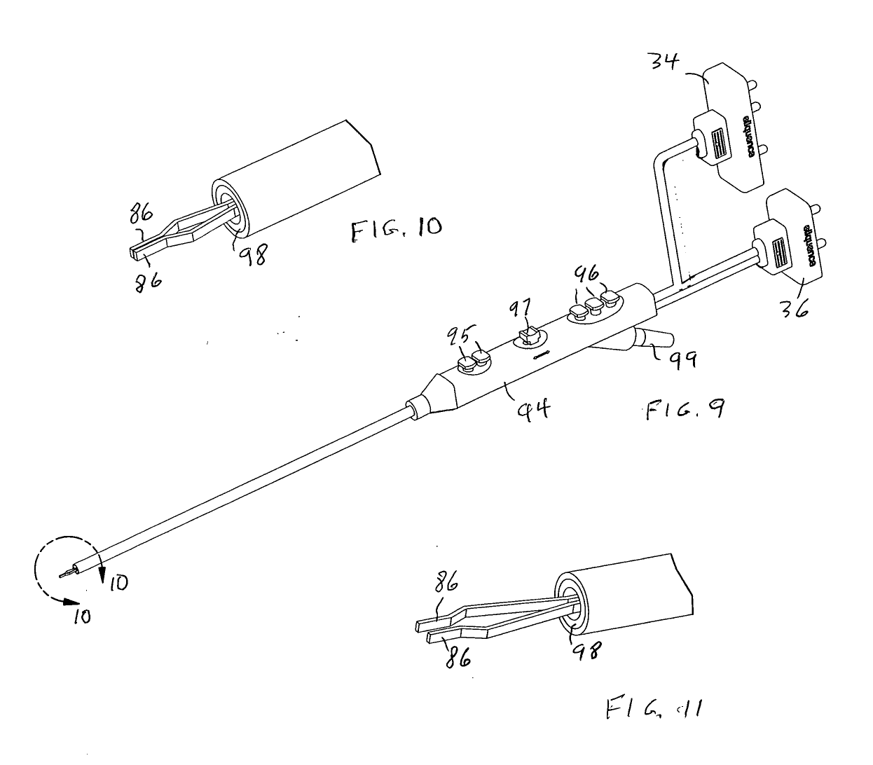

FIGS. 9-11 show a further variant in which the handles used to extend and retract the electrodes serving both monopolar and bipolar functions are replaced by a slide structure similar to that described in connection with FIGS. 18 and 19 of the '571 patent, in combination with fingerswitches to select the electrosurgical action. The handpiece 94 in FIG. 9 has two button fingerswitches 95 connected to supply signals to the electrosurgical unit to activate the bipolar output connector with two variations of bipolar energy, and has three button fingerswitches 96 connected to supply signals to the electrosurgical unit to activate the monopolar output connector with three variations of monopolar energy. The functions of the fingerswitches can be reversed, and fewer fingerswitches can also be used. The slide 97 mounted in the center is connected to the electrodes 86 such that when partially extended as shown in FIG. 10, the sides abut forming a single edge good for monopolar cutting, for example. When the slide is pushed forward, as shown in FIG. 11, the electrode tips separate and can then be used to grab tissue for, for example, coagulation or ablation. See also the '838 application for more details. The two plugs 34, 36 connect as before to the two output connectors 16, 18. In this case, the signals produced by depression of the fingerswitches are directed via the two terminals of the monopolar connector to the unit's internal circuitry to select the desired function, as explained in the referenced applications/patents. FIGS. 10 and 11 also show the extra internal channel 98 connected to the suction fitting 99 to which the suction hose can be connected. Thus this embodiment combines in one pencil-slim handpiece the ability to supply to its electrode tips monopolar energy or bipolar energy with manipulation of the electrode tips to form tissue cutting or tissue grabbing functions in the presence of suction at the electrode distal end to remove undesired materials.

It will be understood that the above examples are only preferred examples and other ways of storing and accessing information representing operating conditions of the system unit or of activating desired known functions of such systems can be employed.

While the invention has been described in connection with preferred embodiments, it will be understood that modifications thereof within the principles outlined above will be evident to those skilled in the art and thus the invention is not limited to the preferred embodiments but is intended to encompass such modifications.

What is claimed is:

1. An electrosurgical instrument having a plurality of operating modes, comprising:
(a) a console unit having:
i) a monopolar output connector supplying monopolar electrosurgical currents when the unit is energized,
ii) a bipolar output connector supplying bipolar electrosurgical currents when the unit is energized,
(b) a handpiece connected to both the unit's monopolar output connector and the unit's bipolar output connector,
(c) first means for selectively energizing the unit to activate the monopolar output connector or the bipolar output connector to supply monopolar or bipolar electrosurgical currents, respectively, to the handpiece,
(d) an electrode mounted in the handpiece, the electrode having a first position configured to perform an electrosurgical cutting action when supplied with monopolar electrosurgical currents and a second position configured to perform a coagulation action when supplied with bipolar electrosurgical currents.

2. An electrosurgical instrument as claimed in claim 1, further comprising a suction fitting connected to the handpiece for supplying suction at the distal end of the handpiece.

3. An electrosurgical instrument as claimed in claim 1, further comprising a neutral plate connected to the instrument for grounding a patient.

4. An electrosurgical instrument as claimed in claim 3, wherein the first means comprises a footswitch connected to the unit and operable by a user into a first position wherein it energizes the unit to activate the monopolar output connector, or into a second position wherein it energizes the unit to activate the bipolar output connector.

5. An electrosurgical instrument as claimed in claim 3, wherein the first means comprises a first fingerswitch connected to the handpiece and operable by a user and connected to energize the unit to activate the monopolar output connector, and a second fingerswitch connected to the handpiece and operable by the user and connected to energize the unit to activate the bipolar output connector.

6. An electrosurgical instrument as claimed in claim 3, wherein the monopolar output connector comprises a first terminal that is electrically active at a given polarity when the monopolar connector is activated, and the bipolar output connector comprises second and third terminals that are electrically active of which the second terminal is at the same given polarity when the bipolar connector is activated, further comprising a cable connected to the handpiece and having a bifurcated end for connecting to both the monopolar output connector and the bipolar output connector, the cable interconnecting both the first and second terminals.

7. An electrosurgical instrument as claimed in claim 6, further comprising connected to the cable first and second connectors adapted to mate with the monopolar output connector and the bipolar output connector, respectively.

8. An electrosurgical instrument as claimed in claim 3, wherein the electrode comprises separable tips, and the handpiece comprises a squeezable handle connected to the electrode such that in a first position of the handle the separable tips are together and in a second position of the handle the separable tips are spaced apart.

9. An electrosurgical instrument as claimed in claim 3, wherein the electrode comprises separable tips, and the handpiece comprises a slide structure connected to the electrode such that in a first position of the slide the separable tips are together and in a second position of the slide the separable tips are spaced apart.

10. An electrosurgical instrument having a plurality of operating modes, comprising:
(a) a console unit having:
i) a monopolar output connector supplying monopolar RF electrosurgical currents when the unit is energized,
ii) a bipolar output connector supplying bipolar RF electrosurgical currents when the unit is energized,
(b) a single handpiece connected to both the unit's monopolar output connector and the unit's bipolar output connector,
(c) first means for selectively energizing the unit to activate the monopolar output connector or the bipolar output connector to supply monopolar or bipolar electrosurgical currents, respectively, to the handpiece.

11. An electrosurgical instrument as claimed in claim 10, wherein the console unit comprises RF generators and modulating means, and the first means comprises a footswitch connected to the unit or fingerswitches on the handpiece connected when operated to activate the RF generators and modulating means for supplying selectively monopolar or bipolar electrosurgical currents to the monopolar output connector or the bipolar output connector, respectively.

12. An electrosurgical instrument as claimed in claim 10, wherein the handpiece comprises a suction fitting to supply suction at the distal end of the handpiece.

13. An electrosurgical instrument as claimed in claim 11, wherein the handpiece comprises bipolar electrodes configured to extend or retract from the handpiece under control of a user.

14. An electrosurgical instrument as claimed in claim 13, wherein the bipolar electrodes upon extension from the handpiece in one position are separated and in another position are in contact.

15. An electrosurgical instrument as claimed in claim 10, wherein the handpiece comprises a cable with conductors connected to a first bipolar connector and a second monopolar connector, one of the conductors being common to both connectors.

16. A method of operating electrosurgical apparatus comprising:
A. providing an electrosurgical instrument having a plurality of operating modes, comprising:
(a) a console unit having:
i) a monopolar output connector supplying monopolar RF electrosurgical currents when the unit is energized,
ii) a bipolar output connector supplying bipolar RF electrosurgical currents when the unit is energized,
(b) a single handpiece connected to both the unit's monopolar output connector and the unit's bipolar output connector,
(c) first means for selectively energizing the unit to activate the monopolar output connector or the bipolar output connector to supply monopolar or bipolar electrosurgical currents, respectively, to the handpiece,
comprising the steps:
B. operating the first means to supply monopolar electrosurgical currents to the handpiece to perform a cutting action on tissue,
C. then operating the first means to supply bipolar electrosurgical currents to the handpiece to perform with the same handpiece coagulation of tissue bleeders.

17. A method as claimed in claim 16, wherein the electrodes under control of a user can be made to abut or to separate.

18. A method as claimed in claim 17, wherein the electrodes are extended to separate and retracted to abut.
